(19) United States
(12) Patent Application Publication
(10) Pub. No.: US 2025/0325371 A1
(43) Pub. Date: Oct. 23, 2025

(54) Method and System for Performing Mitral and Tricuspid Annuloplasty

(71) Applicant: Jose A Condado, Caracas (VE)

(72) Inventor: Jose A Condado, Caracas (VE)

(21) Appl. No.: 19/186,994

(22) Filed: Apr. 23, 2025

Related U.S. Application Data
(60) Provisional application No. 63/637,596, filed on Apr. 23, 2024.

(51) Int. Cl. A61F 2/24; A61B 17/04; A61B 17/32; A61F 2/01; A61M 25/06; A61M 25/09

(52) U.S. Cl. CPC A61F 2/2466; A61B 17/320016; A61F 2/014; A61B 2017/0409; A61B 2017/0464; A61M 25/0662; A61M 2025/09183

(57) ABSTRACT

A method for performing a mitral or tricuspid annuloplasty and forming a support system within the heart is disclosed. The method includes puncturing, using a guidewire from a catheter, a center of a posterior sulcus from the left ventricle to the left atrium to create a central support point. The guidewire is then captured in the left atrium using an attachment device of a second catheter. Additional punctures are performed on each side of the central support point, followed by the attachment of anchor devices with ropes at each of the puncture sites. The ropes are deployed and interconnecting bars are released and attached to the ropes. The bars are then coupled using a zipper mechanism to constrict the annulus. The anchor devices, ropes, and interconnecting bars together form a support system that defines a stable axis spanning the mitral annulus and the interatrial septum.

### RELATED APPLICATIONS

This application claims priority to provisional patent application 63/637,596, filed Apr. 23, 2024, entitled “Method and System for Performing Mitral and Tricuspid Annuloplasty,”

### FIELD OF INVENTION

Various embodiments of the disclosure relate to performing a mitral annuloplasty. More specifically, various embodiments of the disclosure relate to the method and system for performing mitral and tricuspid annuloplasty.

### BACKGROUND

Generally, damage to or malfunctioning of a heart valve of a patient leads to an imbalance in the blood flow in the body of the patient. Further, there are diseases such as mitral regurgitation which is a type of heart valve disease causing blood flow problems. When the patient suffers from mitral regurgitation, a mitral valve of the heart fails to sufficiently close, and the blood is allowed to backflow across the heart valve.

Currently, various surgical techniques are used to repair damaged heart valves. The mitral annuloplasty method is one of the effective surgical techniques used for repairing damaged heart valves. The mitral annuloplasty method comprises reducing the size of a valve annulus by attaching an annuloplasty ring to an interior wall of the heart around the valve annulus. Further, the mitral annuloplasty method is performed for correcting a mitral leak. However, the existing mitral annuloplasty methods are often time-consuming and inefficient: if an improper size of the annuloplasty ring is selected in the mitral annuloplasty method, then the patient may face post-operative complications and suboptimal outcomes. Thus, the existing mitral annuloplasty method is dependent on the accurate selection of the annuloplasty ring size.

In light of the foregoing, there exists a need for technical and reliable solutions that overcome the above-mentioned problems, challenges, and short-comings, and continues to facilitate an end-to-end process required to perform a mitral annuloplasty method.

### SUMMARY

In one embodiment of the present disclosure, a method for performing a mitral or tricuspid annuloplasty and creating a support system within a heart from which other medical procedures may be performed, is provided. The method includes puncturing, using a guidewire from a catheter, a center of a posterior sulcus from a left ventricle to a left atrium, and creating a central support point. Further, the method includes capturing the guidewire in the left atrium with an attachment device of a second catheter. Furthermore, the method includes performing a puncture, using the guidewire, on each side of the central support point. Subsequently, the method includes attaching anchor devices with ropes on each side of each of the punctures. The method further includes deploying the ropes attached to the anchor devices. The method includes releasing interconnecting bars attached to the ropes. Finally, the method includes coupling the interconnecting bars using a zipper. The anchor devices, the ropes, and the interconnecting bars form a support system with a stable axis that incorporates both a mitral annulus and an interatrial septum.

Additionally, or optionally, the method comprises placing a guide catheter in the left atrium by atrial transseptal puncture and in the left ventricle by an arterial route from an aorta of the heart.

Additionally, or optionally, the step of puncturing further comprises first puncturing the center of a posterior semicircle of the mitral annulus, which corresponds to a P2 segment of a mitral valve, and wherein a central catheter is used for the puncture and comprises two catheters.

Additionally, or optionally, the attachment device of the second catheter is a telescoping system of a loop catheter, and the step of capturing further comprises trapping a guide tip in the left atrium with the telescoping system of the loop catheter.

Additionally, or optionally, the second catheter capture attachment device comprises a system of baskets, and wherein the baskets are introduced via a venous catheter through auricular transeptal.

Additionally, or optionally, performing a puncture, using the guidewire, on each side of the central support point comprises making lateral punctures at a constant distance from the support point using a central support catheter and a catheter parallel to the central support catheter.

Additionally, or optionally, the anchor devices comprise screws and the step of attaching anchor devices with ropes on each side of the punctures comprises screwing to fix to the anchor devices.

Additionally, or optionally, deploying the ropes attached to the anchor devices comprises pulling the ropes.

Additionally, or optionally, the step of releasing the interconnecting bars attached to the ropes comprises unfolding the interconnecting bars as they exit the catheter on the side of the left atrium, and wherein the unfolded interconnecting bars are attached to one another and are prevented from separating.

Additionally, or optionally, the step of coupling the interconnecting bars using the zipper comprises constricting the mitral annulus with the bars.

In another embodiment, a system for performing a mitral or tricuspid annuloplasty and creating a support system within a heart from which other medical procedures may be performed is provided. The system includes trans-atrial guide catheters. The system further includes an arterial retrograde catheter having a metallic guidewire including a tip. The metallic guidewire is configured to puncture a center of a posterior sulcus from a left ventricle to a left atrium creating a central support point. After puncturing, the metallic guidewire tip is captured in the left atrium with an attachment device of a second catheter. The metallic guidewire is configured to perform a puncture on each side of the central support point. The system also includes anchor devices. The system further includes ropes attached to the anchor devices. The anchor devices with ropes are attached on each side of the punctures. The system further includes interconnecting bars attached to the ropes. The interconnecting bars are coupled using a zipper. The anchor devices, the ropes, and the interconnecting bars form the support system, connected to the central support point, with a stable axis that incorporates both a mitral annulus and an interatrial septum.

Various embodiments of the present disclosure provide a method and a system for performing a mitral annuloplasty, creating a support system within a heart from which other medical procedures may be performed, and all the components of performing the mitral annuloplasty that is not possible to perform using existing techniques.

### BRIEF DESCRIPTION OF THE DRAWINGS

FIG. 1 is a process flow diagram of a method for performing a mitral or tricuspid annuloplasty and creating a support system within a heart from which other medical procedures may be performed, in accordance with an embodiment of the present disclosure;

FIG. 2 shows a mitral annuloplasty approach method comprising an intravascular loop installation from the femoral vein via transseptal-to-atrial route to the femoral artery, in accordance with an embodiment of the present disclosure;

FIG. 3 shows a basket catheter and a magnetic support catheter for puncturing in valvular ring, in accordance with an embodiment of the present disclosure;

FIG. 4 shows a telescoping system of a loop catheter for trapping a guidewire, in accordance with an embodiment of the present disclosure;

FIG. 5 shows making lateral punctures point using a central support catheter and a catheter parallel to the central support catheter, in accordance with an embodiment of the present disclosure;

FIG. 6 shows ventricular screw anchors integrated with interconnector, in accordance with an embodiment of the present disclosure;

FIG. 7 shows mitral annuloplasty with central interconnector grouping the guide chords in the center and maintaining lateral chords, in accordance with an embodiment of the present disclosure;

FIG. 8 shows ribbon interlock with zipper, in accordance with an embodiment of the present disclosure; disclosure;

FIG. 9 shows a support system, in accordance with an embodiment of the present

FIG. 10 shows anchor models, in accordance with an embodiment of the present disclosure; and

FIG. 11 shows integration of the central occluder without central disk, in accordance with an embodiment of the present disclosure.

### DETAILED DESCRIPTION

Exemplary systems and related methods are described herein. Other exemplary embodiments or features may further be utilized, and other changes may be made, without departing from the spirit or scope of the subject matter presented herein. In the following detailed description, reference is made to the accompanying drawings, which form a part thereof. Accordingly, the exemplary embodiments described herein are not meant to be limiting. It will be readily understood that the aspects of the present description, as generally described herein, and illustrated in the drawings, can be arranged, substituted, combined, separated, and designed in a wide variety of different configurations, all of which are explicitly contemplated herein. Similar features and components of the disclosed implementations include the hundred-place numeral of the corresponding Figure, as appropriate, in which:

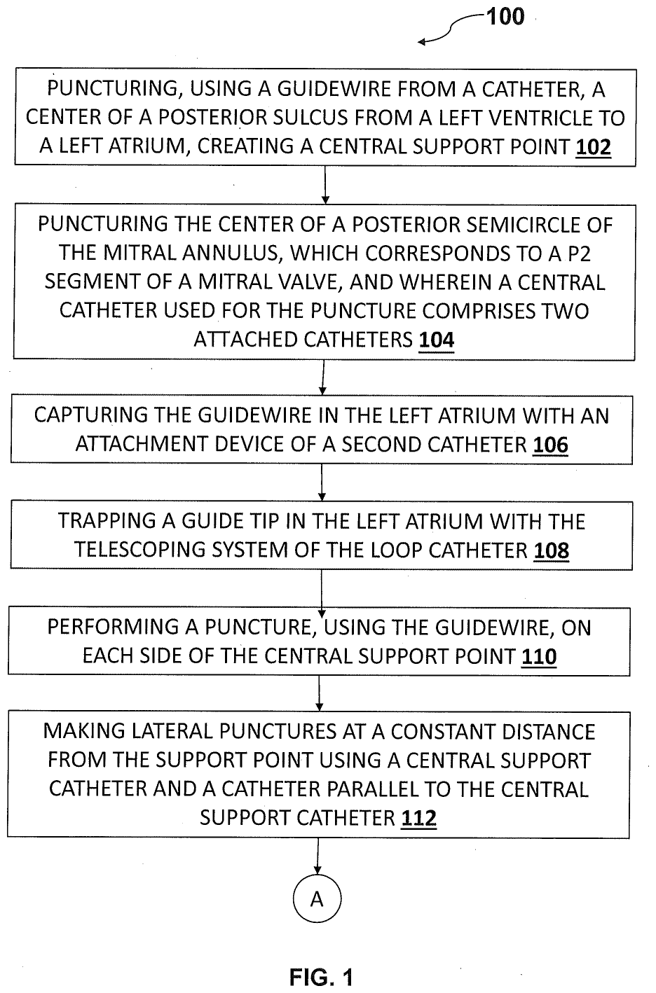

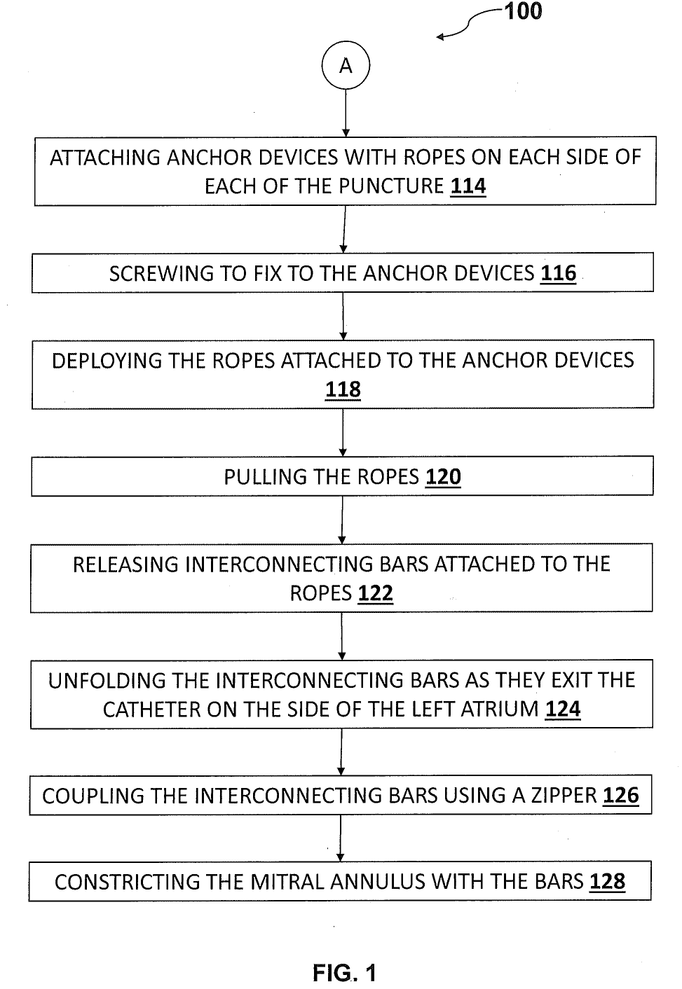

FIG. 1 is a process flow diagram of a method for performing a mitral or tricuspid annuloplasty and creating a support system within a heart from which other medical procedures may be performed, in accordance with an embodiment of the present disclosure.

The method 100 comprises, at step 102 puncturing, using a guidewire from a catheter, a centre of a posterior sulcus from a left ventricle to a left atrium, creating a central support point. In one embodiment, this involves deploying a support catheter through an arterial guiding catheter placed retrogradely from an aorta into the left ventricle. The support catheter includes metal guides and is configured to perform a puncture at the centre of the posterior semicircle of the mitral annulus, with the intention of passing through the muscle at the base, (not at the base of the posterior leaflet, because it is very fragile and susceptible to tearing and rupture with traction). The puncture is performed in the posterior sulcus of the mitral valve, and the metallic guidewire is advanced through the muscle and annular tissue until it is exposed in the left atrium, guided by fluoroscopy and ultrasound (TT, TE, and IC) imaging. A curvature of the catheter is configured to maintain a stable position in the sulcus, compensating for cardiac motion.

In an embodiment, at step 104, the step of puncturing the centre of the posterior semicircle (sulcus) of the mitral annulus, corresponding to the P2 segment of the mitral valve, is performed using a central catheter comprising two catheters (a dual catheter) attached side-by-side at the tip. The tip includes radiological metallic markings to aid visualization during fluoroscopy. This dual-catheter assembly allows the controlled exit of the metallic guidewire used to perform the puncture through the annulus. The guidewire is advanced under the guidance of transoesophageal and intracardiac ultrasound, with the catheter positioned to maintain stable and constant contact with the posterior sulcus despite cardiac motion. The puncture step 102 establishes the central support point critical for symmetric anchoring and subsequent procedural steps. The central catheter system also integrates with lateral catheters for additional punctures. However, the initial central puncture at the P2 location ensures that the subsequent device deployment aligns with the natural anatomy of the mitral annulus, offering structural balance and optimal positioning for annuloplasty and prosthetic interventions.

At step 106, the method 100 includes capturing the guidewire in the left atrium with an attachment device of a second catheter. The second catheter is introduced through a venous guiding catheter via a transseptal route into the left atrium and comprises a basket or metallic loop system configured to trap the guidewire tip as the guidewire tip emerges in the left atrium. The captured guidewire is then externalized through the venous guiding catheter, thereby forming a metallic arteriovenous loop, which serves as a stable transport axis across the mitral annulus.

In an embodiment, at step 108, the step of capturing further comprises trapping a guide tip in the left atrium with a telescoping system of a loop catheter. The loop catheter, in conjunction with a telescoping shaft, allows for precise snaring and securing of the guide tip that emerges into the left atrium from the transseptal puncture, enabling the alignment and stabilization of the guidewire, which is essential for the accurate deployment of anchoring and support structures in subsequent steps of the procedure.

At step 110, the method 100 includes performing a puncture, using the guidewire, on each side of the central support point. The puncture is performed by using lateral catheters positioned parallel to the central catheter, each containing metallic guides. The metallic guides are advanced through multiple side holes located along the catheter's contact surface with the posterior sulcus. The metallic guides are extended at a first distance, for example, 10, 15, or 20 mm, laterally from the central puncture point to perform additional perforations on both sides. The catheters are curved to direct the exit angle of the guides for precise lateral punctures, maintaining consistent spacing from the central support point.

In an embodiment, at step 112 performing the puncture, using the guidewire, on each side of the central support point comprises making lateral punctures at a constant distance from the support point using a central support catheter and a catheter parallel to the central support catheter, ensuring symmetrical anchor positioning and uniform force distribution around the mitral annulus, which is helpful for effective annular constriction and proper valve leaflet coaptation.

At step 114, the method 100 comprises attaching anchor devices with ropes on each side of each of the punctures. After all punctures are made, anchor devices are introduced through the arterial catheter and positioned on the ventricular side, while corresponding retrograde anchors are introduced via the venous catheter to fixate on the posterior auricular wall. Each anchor device is tied to a cord or rope which is passed through the guidewire path. Anchors are placed progressively, centrally first, then laterally, on both sides. The anchoring provides a broad area of apposition to reduce the cutting effect during traction and ensures stability during cardiac motion.

In an embodiment, at step 116, the step of attaching anchor devices with ropes on each side of each of the punctures comprises screwing to fix to the anchor devices. The ropes are threaded and rotated into engagement with the anchor devices, such that a threaded or helical feature on the rope or the anchor provides secure fixation in the annular tissue, enhancing mechanical stability and preventing anchor dislodgment under cardiac loading conditions.

At step 118, the method 100 includes deploying the ropes attached to the anchor devices. In one embodiment, deploying the ropes involves pulling the ropes that are already connected to the anchors, extending the ropes across the punctured paths. The ropes may be made of, for example, nylon and/or Gore-Tex and/or other biocompatible synthetic material such as dacron and polyvinyl. The ropes are passed through tunnels or fixation points in the anchor bodies. Once deployed, the ropes establish traction between ventricular and atrial anchors on both sides of the central puncture.

In an embodiment, at step 120, deploying the ropes attached to the anchor devices comprises pulling the ropes to establish and maintain tension between the anchor points on the ventricular and atrial sides. Once the ropes are connected to the anchors, the ropes are pulled manually or via catheter-based tensioning mechanisms to draw the anchors toward one another. The tension between the anchor points ensures the anchoring system is securely seated within the posterior sulcus and the opposing atrial wall, reducing slack and aligning the support structure along the mitral annulus.

The traction applied to the ropes allows for a constrictive force to be transmitted across the posterior segment of the annulus, aiding in approximating the posterior and anterior mitral valve leaflets. The pulling step may also be used to test anchor stability and to position the ropes correctly prior to attaching the interconnecting bars, ensuring that the deployed ropes are functionally integrated into the support framework, forming the foundational tension required for the subsequent bar coupling and zipper-based constriction of the annular segment.

At step 122 the method 100 comprises releasing interconnecting bars attached to the ropes. The interconnecting bars are loaded over the ropes and are delivered through the guide catheters to the atrial side. As the interconnecting bars exit the catheter, the interconnecting bars unfold or expand, depending on the configuration. The interconnecting bars are connected to the ropes and positioned across the mitral annular segment. The interconnecting bars at each end are deployed first, followed by intermediate connectors, as needed. The interconnecting bars serve to distribute traction forces evenly and reduce localized pressure that could cause tissue damage.

In an embodiment, at step 124, the step of releasing the interconnecting bars attached to the ropes comprises unfolding the interconnecting bars as they exit the catheter on the side of the left atrium, and wherein the unfolded interconnecting bars are attached to one another and are prevented from separating. The design of the bars includes locking mechanisms or interlocking geometries that ensure their secure coupling upon deployment, thereby creating a stable continuous support structure along the annular plane.

At step 126, the method 100 comprises coupling the interconnecting bars using a zipper. In one embodiment, the interconnecting bars are equipped with a zipper or hook system that enables them to be fastened together after deployment, forming a continuous annular support structure. The zipper coupling causes a constrictive effect on the mitral annulus, reducing the annular diameter and improving the apposition between anterior and posterior leaflets of the mitral valve.

In an embodiment, at step 128, the step of coupling the interconnecting bars using the zipper comprises constricting the mitral annulus with the bars. The zipper draws the bars together, applying circumferential tension that reduces the annular diameter and reshapes the mitral valve to restore functional leaflet coaptation and mitigate regurgitation.

Further, the anchor devices, the ropes, and the interconnecting bars form a support system with a stable axis that incorporates both the mitral annulus and the interatrial septum. The anchor and bar system ensures a fixed reference that can also serve as a platform for deploying a mitral clip, posterior leaflet support, mitral valve prosthesis and/or other items as required. The support frame may additionally interact with a double-disc septal support system for interatrial septum anchoring, ensuring further stability.

Furthermore, the method 100 includes placing a guide catheter in the left atrium by atrial transseptal puncture and in the left ventricle by an arterial route from the aorta. Specifically, a venous guiding catheter (for example, 18-24 French (Fr)) is introduced via the right femoral vein and advanced through a mid-to-high transseptal puncture into the left atrium. Separately, a retrograde arterial guiding catheter (for example, 16 Fr) is introduced through the left femoral artery, across the aortic valve and into the left ventricle. Together, the guide catheters establish a working axis across the mitral valve by interconnecting through the metallic guidewire that traverses from the left ventricle to the left atrium, creating an arteriovenous loop. Such a stable loop forms the procedural backbone through which catheters and devices are safely deployed without compromising valve function, ventricular contractility, and/or coronary flow.

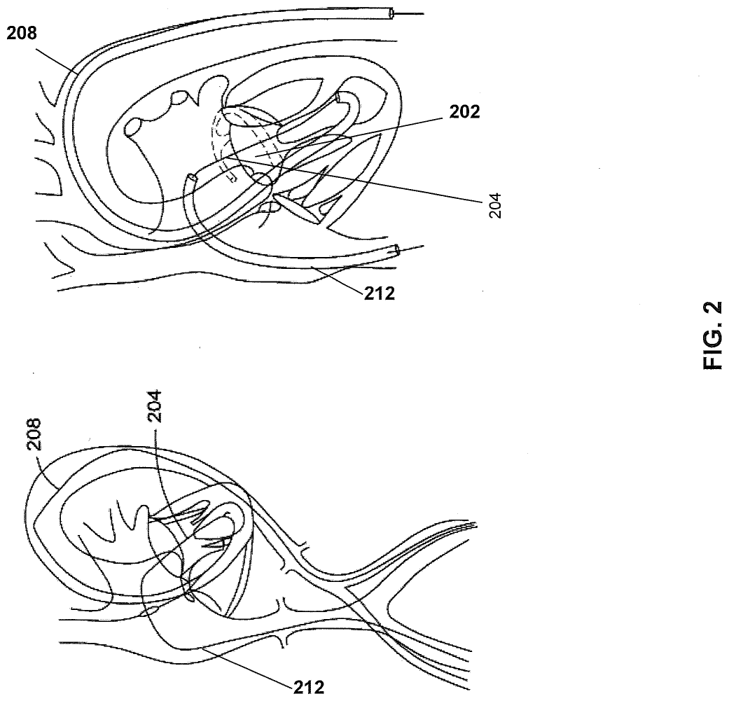

FIG. 2 shows a mitral annuloplasty approach method comprising an intravascular loop installation from the femoral vein via transseptal-to-atrial route to the femoral artery, in accordance with an embodiment of the present disclosure. In an embodiment, under sedation and anticoagulation, and with continuous pressure monitoring and transesophageal echocardiographic guidance, a right femoral vein is cannulated, and a venous guiding catheter (for example, 18-24 Fr) 212 is introduced and advanced to the left atrium via a mid-to-high transseptal puncture 906 Simultaneously, a left femoral artery is cannulated, and a retrograde arterial guiding catheter (for example, 16 Fr) 208 is introduced through the aorta into the left ventricle.

Further, a balloon catheter is advanced from the venous guiding catheter 212 traversing the left atrium, mitral valve 202 and left ventricle, and into the descending aorta using a hydrophilic guidewire. The hydrophilic guidewire is then exchanged for a stiffer guidewire (for example, 0.035″, 400 cm), which is positioned securely in the descending aorta. In one embodiment, a catheter with a metallic loop or basket is then introduced through the arterial guide 208 used to trap and capture the support guidewire in the aorta. Once captured, the support guidewire is externalized through the arterial introducer, thereby forming a metallic arteriovenous loop 204 that runs from the right femoral vein, across the interatrial septum, through the left atrium and ventricle, and out via the left femoral artery. Such an intravascular loop forms a stable working axis through the heart, specifically across the mitral valve 202 and allows for the controlled delivery of annuloplasty system components, such as catheters, anchors, ropes, and interconnecting bars. The arteriovenous loop 204 facilitates a central alignment of devices with the posterior segment of the mitral annulus, particularly at the P2 segment, ensuring procedural safety and precision while minimizing interference with valve function, coronary flow, or ventricular contraction.

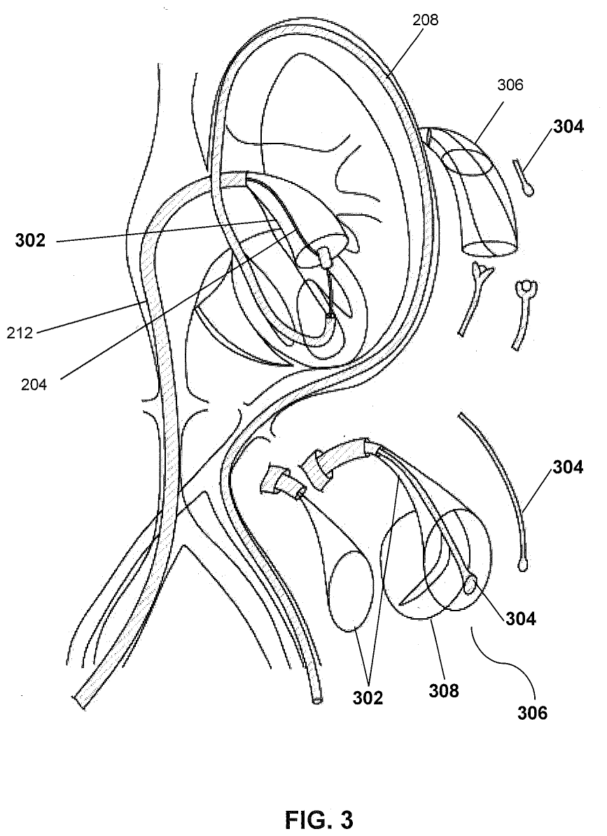

FIG. 3 shows a basket catheter 302 and a magnetic support catheter 304 for puncture in valvular ring, in accordance with an embodiment of the present disclosure. In an embodiment, a second catheter comprises a capture attachment device in the form of a system of baskets 306 The system of baskets 306 comprises a basket catheter 302 and a magnetic support catheter 304 The system of baskets 306 is configured for trapping the guidewire tip within a cardiac chamber, specifically within the left atrium, after the guidewire has punctured through the mitral annulus from the left ventricle. The system of baskets 306 may be introduced via a venous catheter 212 through an auricular transseptal route, enabling precise positioning and control within the left atrium. The system of baskets 306 aids in securing the guidewire tip once exposed in the left atrium, thereby assisting in forming a central support point that serves as a reference for additional procedural steps.

Further, the system of baskets 306 of the second catheter may be either symmetrical or asymmetrical, allowing for various configurations depending on the anatomical geometry and procedural requirements. The symmetry or asymmetry of the system of baskets 306 ensures enhanced maneuverability and the ability to conform to complex atrial geometries during deployment. In one implementation, the basket catheter 302 may be integrated with a metallic loop 308, forming a combined capture mechanism for the guidewire, which helps establish a stable arteriovenous loop 204 used as a transport axis for catheter-based interventions.

The system of baskets 306 comprising the basket catheter 302 and the magnetic support catheter 304 enables the capture of the guidewire tip in the left atrium without displacing or dislodging it from its punctured position, and contributes to the stabilization of the central axis that incorporates both the mitral annulus and the interatrial septum. The central axis is useful for the subsequent steps of anchor placement, rope deployment, and interconnecting bar attachment.

Additionally, the use of the venous guide catheter 212 for basket delivery via the auricular transeptal route minimizes procedural invasiveness while offering high accessibility to the left atrium. Such an arrangement also allows for externalization of the guidewire through the venous guide catheter 212 which is connected to the arterial guidewire to form the metallic atriovenous loop 204, thus establishing a transport path for delivering the annuloplasty system components.

Accordingly, the second catheter with a system of baskets 306 provides an effective mechanism for guidewire capture and stabilization, which is useful to ensure the precision of device deployment, especially in dynamically moving cardiac environments.

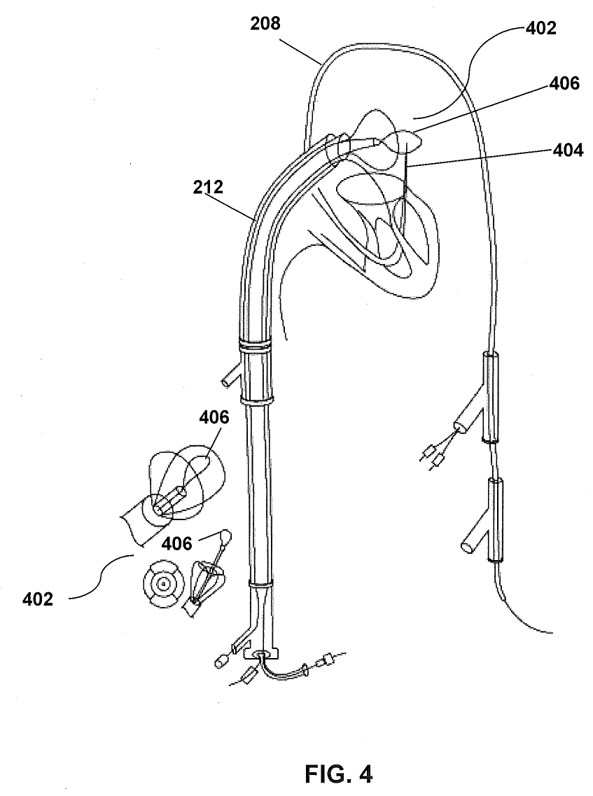

FIG. 4 shows a telescoping system 402 of a loop catheter for trapping a guidewire 404, in accordance with an embodiment of the present disclosure. In one embodiment, a second catheter includes an attachment device configured as a telescoping system 402 of a loop catheter. The telescoping system 402 is deployed via a venous transseptal route into the left atrium, where it is used to trap the guidewire tip that has punctured from the left ventricle through the mitral annulus and entered the left atrium. The telescoping system 402 enables precise capture of the guidewire 404 while maintaining alignment with the central axis of the heart chamber.

The telescoping system 402 includes slidable, nested components that extend and contract to conform to the spatial orientation of the guidewire 404 The loop at the distal end 406 of the telescoping system 402 is configured to encircle and trap the guide tip as it emerges through the mitral annular puncture into the left atrial cavity. The telescoping system 402 enables secure engagement of the guidewire 404 while preserving the positional integrity of the working axis between the mitral annulus and the interatrial septum.

The telescoping system 402 is particularly advantageous in dynamic cardiac environments, as the telescoping system 402 provides flexibility during guidewire capture and allows for controlled manipulation during the formation of the metallic arteriovenous loop 204, which is important for subsequent annuloplasty procedures.

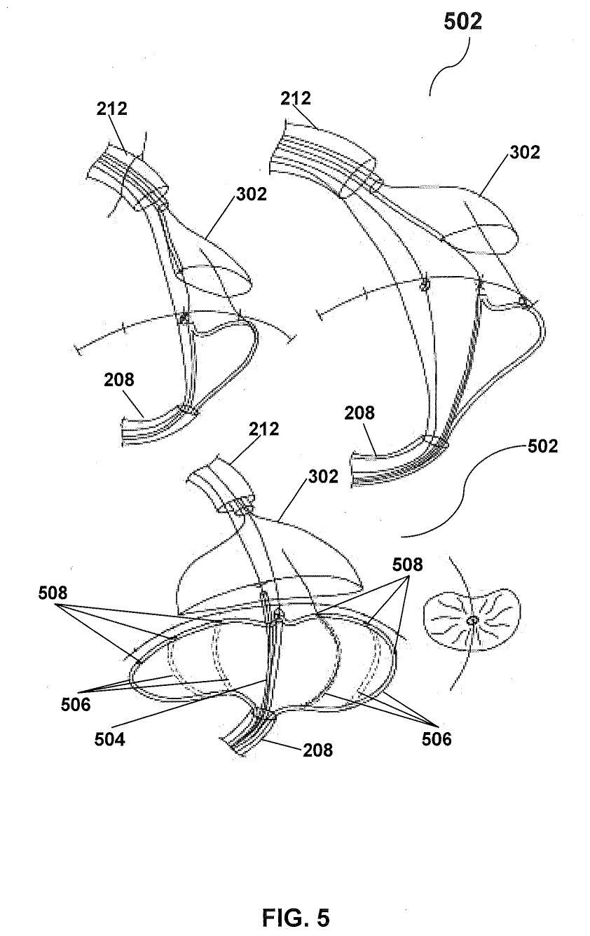

FIG. 5 shows making lateral punctures 502 using a central support catheter 504 and a catheter parallel 506 to the central support catheter 504, in accordance with an embodiment of the present disclosure. After establishing the central puncture at the posterior sulcus of the mitral annulus using the central support catheter 504, additional lateral punctures 502 are created on each side to facilitate multi-point anchoring. Multi-point anchoring is achieved by placing one or more curved catheters 506 parallel to the central support catheter 504. These curved lateral catheters 506 are equipped with side ports or windows 508 located along the portion of the catheter in contact with the posterior sulcus, allowing for controlled deployment of metallic guides or needles.

The lateral punctures 502 are spaced at predetermined distances, typically 10 mm, 15 mm, or 20 mm, from the central puncture, enabling symmetrical placement of anchor points on either side of the central axis. The distance between punctures can vary. In some embodiments the distances between punctures can be non-uniform, for example 3 mm, 8 mm, and 13 mm. The punctures can vary in the range of approximately 3 mm to 25 mm. The curved design of the catheters ensures that the exit trajectory of the guides remains tangential to the annular contour, allowing for accurate penetration of the tissue without damaging surrounding structures. Such multiple puncture points form the basis for installing anchor devices and routing ropes, which ultimately contribute to a uniform, tensioned support system for annular constriction and reinforcement.

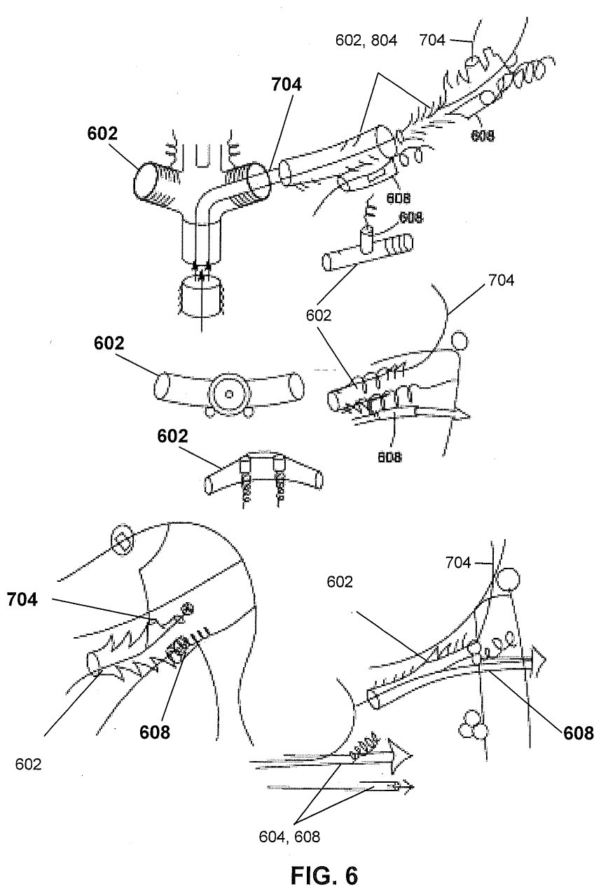

FIG. 6 shows ventricular screw anchors 608 integrated with interconnector 602 in accordance with an embodiment of the present disclosure. In one embodiment, the anchor devices 604 attached to each side of the punctures comprise ventricular screw anchors 608 The ventricular screw anchors 608 are configured to be screwed into the tissue to achieve secure fixation within the ventricular wall. Each ventricular screw anchor 608 is connected to a rope or cord, which is deployed to apply traction and transmit force between anchoring points across the mitral annulus.

The ventricular screw anchors 60B are integrated with interconnectors 602 that form part of the support framework spanning the mitral annular segment. The interconnectors 602 are pre-attached to the ropes and are positioned during the procedure by advancing them through the catheter system until they exit on the atrial and/or ventricular side. Upon deployment, the interconnectors 602 unfold and are secured at each end to the respective ventricular screw anchors 608, ensuring the interconnectors 602 remain stably fixed in place.

The integration of ventricular screw anchors 608 with interconnectors 602 helps form a robust and stable support structure that distributes the traction forces exerted by the ropes, prevents anchor recoil, and maintains annular constriction. The integration of ventricular screw anchors 608 with the interconnectors 602 further contributes to the stable axis that incorporates the mitral annulus and interatrial septum, as described in earlier steps of the method.

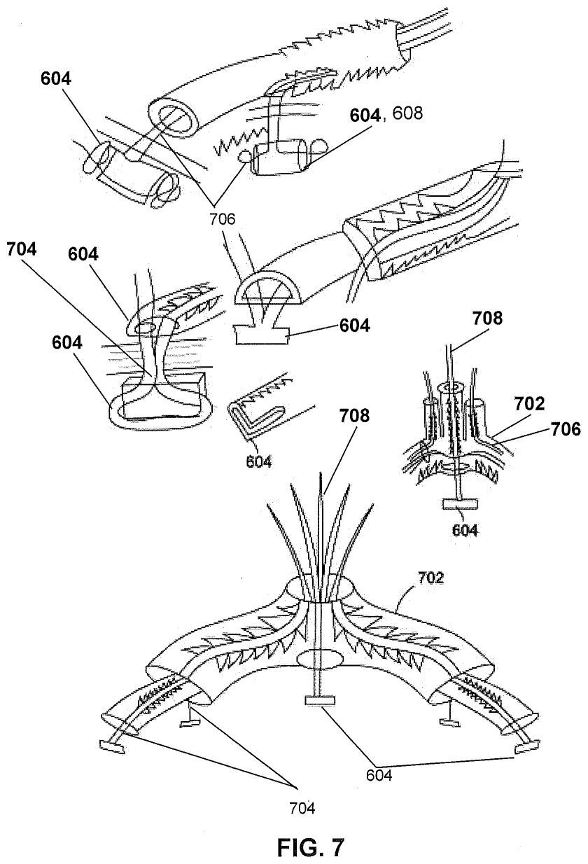

FIG. 7 shows mitral annuloplasty with central interconnector 702 grouping the guide chords 708 in the center and maintaining lateral chords 706, in accordance with an embodiment of the present disclosure. In one embodiment, the step of releasing the interconnecting bars 904 attached to the ropes 704 comprises unfolding the interconnecting bars 904 as they exit the catheter into the left atrium. The interconnecting bars 904 are pre-attached to the ropes 704 and are designed to unfold or expand upon deployment to conform to the annular curvature. The interconnecting bars 904 are configured to group central guide chords 708 into the central interconnector 702 while maintaining the lateral chords 706 on each side in their respective positions.

The unfolded interconnecting bars 904 are then attached to one another, forming a continuous structure across the mitral annulus. The connection between bars 904 is configured such that they are prevented from separating once engaged. Such structural integration and structural integrity ensure that the traction forces transmitted via the ropes 704 are evenly distributed along the interconnecting bars 904, thereby enhancing the stability and durability of the support system. The central grouping 708 of the chords helps to focus the annular constriction effect at the posterior centre (P2 segment), while still anchoring the lateral edges, ensuring comprehensive annular remodelling. The interconnecting bars 904 connect the guides or the ropes 704. Further, the interconnecting bars 904 may have a zipper 806 or hooks that hook between them (“interconnectors” 812), so that once interconnected, the bars are united, tied, and maintain the traction force with a distribution of the support force along the bar (not ripping or cutting).

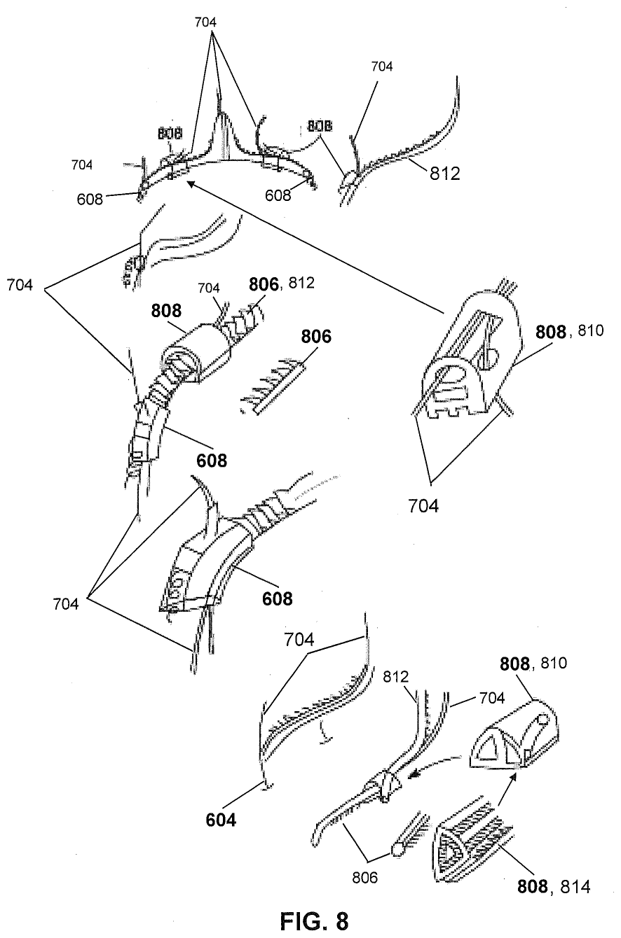

FIG. 8 shows the use of interconnectors 812 and interconnects 808, which include interconnector 602 shown in FIG. 6 and central interconnector 702 shown in FIG. 7, hereafter generally referred to as interconnects 808

The interconnectors 812 may be belts or straps with zippers 806 that have anchors 604 with ropes 608 at the ends and are connected to a tubular interconnect 810 that integrates the ropes 704 at the end with the ropes 704 and/or guide cords at the center 708

Interconnects 808 are devices that connect the ropes 704 and interconnecting bars 904, and in one embodiment placing the interconnects 808 constricts the mitral annulus. Interconnects 808, aka interlocks 808, in one embodiment, are tubular-do not have teeth but have tunnels 810-and in another embodiment, interconnects have teeth, or zippers 814 Some of these interconnects with teeth 814 are further attached to zippers 806, that are attached to anchors 604 with ropes and interconnecting bars 904. In one embodiment, tubular interconnects 810 connect the lateral ropes 706 and central ropes, for example the guide chords 708 shown in FIG. 7. Thus, pulling the ropes 706 tightens the system, as the ropes 704 are attached to the interconnects 808 and/or anchors 604 and/or bars 904. FIG. 8 shows ribbon interlocks with zippers 806, and a tubular interconnector 810, in accordance with an embodiment of the present disclosure. There are two types of interconnectors shown: tubular interconnects 810 and interconnects with teeth 814

In one embodiment, the step of coupling the interconnecting bars 904 using the zipper 806 comprises constricting the mitral annulus by drawing the bars 902 together through the interlocks. Each interconnecting bar 902 is designed to include a zipper interface 804 that allows it to be joined securely with adjacent bars 904 upon deployment.

The ribbon interlock 808 with zipper 806 forms a continuous annular constriction band by “zipping” or coupling the interconnecting bars 904 together, enabling a controlled reduction of the annular diameter. Such a zipper 806 coupling system ensures firm and fixed attachment between the bars 904, preventing separation even under dynamic loading from the cardiac cycle. The constrictive effect improves coaptation of the mitral valve 202 leaflets, particularly between the anterior and posterior segments, and contributes to functional restoration of valve competence.

In accordance with an embodiment, the present disclosure provides a system for performing a mitral or tricuspid annuloplasty and creating a support system within a heart from which other medical procedures may be performed. The system comprises trans-atrial guide catheters introduced through a venous route, such as from the right femoral vein, and advanced to the left atrium via a transseptal puncture 906 The trans-atrial guide catheters are used to deploy basket catheters 302 loop catheters, and delivery tools for the anchor devices 604, the ropes 704, and the bars 904 into the left atrium. The trans-atrial guide catheters 208 are positioned in the left atrium, enabling catheter-based device manipulation without obstructing valve function or coronary flow.

In some embodiments, the system further includes an arterial retrograde catheter 208 inserted via the left femoral artery, advanced retrogradely through the aorta and aortic valve, and positioned in the left ventricle. The metallic guidewire 404, such as, for example, a 0.014-inch ×190 cm wire, is delivered through this arterial catheter 208 and is configured to puncture a centre of a posterior sulcus from the left ventricle to the left atrium, forming a central support point aligned with the P2 segment of the mitral annulus.

In some embodiments, after puncturing, a tip of the metallic guidewire 404 is exposed in the left atrium. There, it is captured using an attachment device of a second catheter, which may include a basket system 306 or a telescoping loop catheter 402 as described in earlier sections and shown in FIG. 4 of the present disclosure. The capture attachment device is introduced via the venous transseptal guide catheter 212 into the left atrium and is designed to trap and secure the guidewire tip, forming a metallic arteriovenous loop 204 when the guidewire 404 is externalized through the venous side.

The metallic guidewire 404 is further configured to perform additional punctures on each side of the central support point, using lateral catheters 506 with metallic guides and pre-positioned side holes 508 The lateral catheters 506 are advanced in parallel to the central catheter and are curved to maintain contact with the posterior sulcus. The guidewire 404 exits through the lateral ports 502 producing multiple punctures spaced for example at one or more of: 5, 10, 15, 20 and/or 25 mm from the central puncture.

In some embodiments, the system includes anchor devices 604 deployed at each puncture point. The anchor devices 604 may be screw-type anchors 608, as shown in FIG. 6, and are inserted into the ventricular groove and/or atrial wall. The anchors 604 include screws 606 to prevent movement from a placed site. The anchors 604 are attached to ropes 704 that traverse the annular tissue through the puncture paths. In one embodiment, each anchor 604 is provided with tunnels 1002 or passages for the ropes 704 to securely pass through the anchor and be fixed in place.

The ropes 704 extend from the ventricular anchor 604 to an atrial anchor 604 or fixator and are deployed by pulling, as described previously, creating a tensioned framework across the mitral annulus. The ropes 704 serve both as a means of traction and as guides for delivering interconnecting bars 904

In some embodiments, the system further comprises interconnecting bars 904 that are attached to the ropes 704 and are delivered through the guide catheters. The interconnecting bars 904 are attached to one another. Upon exit from the catheter, the interconnecting bars 904 unfold or expand and are positioned on the atrial side of the mitral annulus. The interconnecting bars 904 are coupled using a zipper 806, such as shown in FIG. 8. The zipper mechanism facilitates controlled coupling of the bars 802 enabling the annular constriction effect necessary for mitral or tricuspid valve repair.

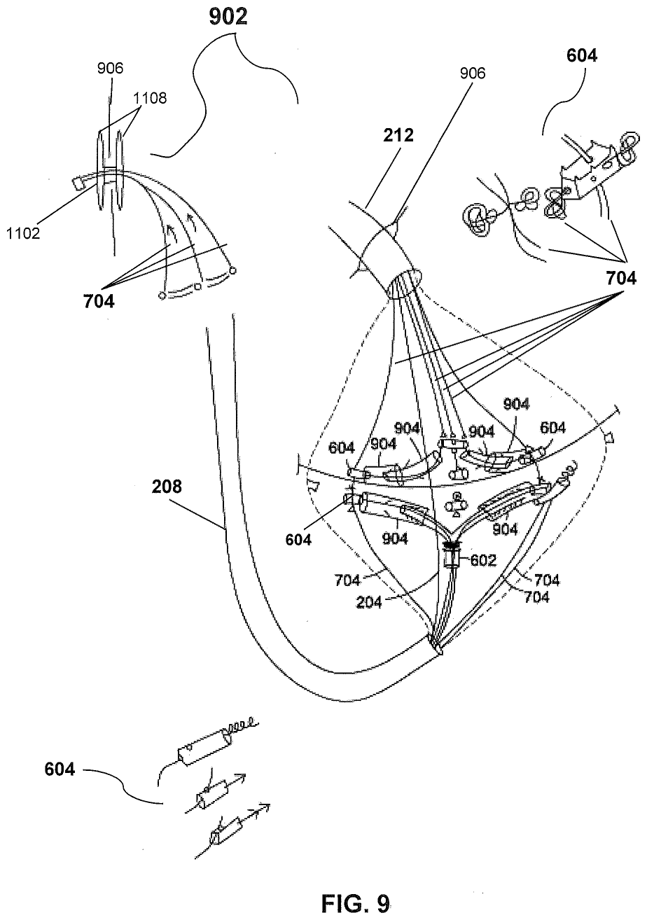

FIG. 9 shows a support system 902 in accordance with an embodiment of the present disclosure. The support system 902 is formed upon the coordinated deployment of multiple components, including anchor devices 604, ropes 704, and interconnecting bars 904, each strategically positioned within and across the cardiac chambers. Initially, anchor devices 604 are implanted at puncture points created along the posterior sulcus of the mitral annulus—one centrally and others laterally on each side. The anchors 604 are securely embedded into the ventricular and atrial walls, with ropes 704 extending through the punctures to bridge each corresponding pair of anchors 604 The ropes 704 are then tensioned to draw the anchors 604 toward each other, establishing the foundation for a robust internal scaffold.

Upon deployment, the interconnecting bars 904 are delivered and attached to the ropes 704 on the atrial side. The interconnecting bars 904 unfold and are joined together using the zipper 806 mechanism, forming a continuous, tensioned structure across the posterior segment of the mitral annulus. The resulting configuration defines a stable anatomical axis that traverses from the ventricular side, through the mitral annulus, and into the interatrial septum, where it can integrate with an optional septal support and/or occluder device 1102 The stable axis serves not only to constrain and remodel the annular geometry but also to provide a predictable and fixed reference platform within the heart.

The support system 902 established by such a configuration is adaptable and multifunctional, as is designed to be compatible with additional therapeutic interventions, including mitral clip implantation, posterior leaflet stabilization, delivery of mitral valve prostheses, and aortic root stabilizations. By maintaining structural integrity and reproducibility in positioning, the support axis improves the safety and precision of such procedures. Furthermore, its placement respects cardiac anatomy and function, minimizing interference with ventricular contraction, valvular mobility, and coronary perfusion, while enhancing the durability and long-term effectiveness of the repair or replacement strategy.

In some embodiments, as described in FIG. 2 and other related descriptions, the system further comprises a metallic atriovenous loop 204 that is interconnected between the trans-atrial guide catheters and the trans-valvular mitral valve 202 The metallic atriovenous loop 204 is formed by first advancing the guidewire 404 from the venous transseptal catheter 212 through the left atrium, across the mitral valve 202 and into the left ventricle, and from there into the descending aorta. The basket 302 or loop catheter, delivered through an arterial guiding catheter 208 introduced via the left femoral artery, is then used to trap the guidewire tip in the descending aorta. The guidewire 404 is subsequently externalized through the arterial introducer, thereby creating a continuous metallic arteriovenous loop 204 between the venous guide catheter 212 and arterial guide catheter 208 This metallic arteriovenous loop 204 forms a stable working axis that traverses the trans-atrial pathway, crosses the interatrial septum by transseptal puncture 906, enters the left atrium, passes through the mitral valve 202 and terminates at the arterial side. Such a configuration enables stable and coaxial delivery of catheters and procedural components, such as anchors 604, ropes 704, and interconnectors 602, by maintaining alignment across the valve and facilitating safe device manipulation along the metallic atriovenous loop 204 path. The metallic arteriovenous loop 204 also ensures that device deployment through the trans-atrial and trans-valvular paths is conducted with high positional accuracy, reducing the risk of malalignment, and helping to preserve valvular and ventricular function during and after the procedure.

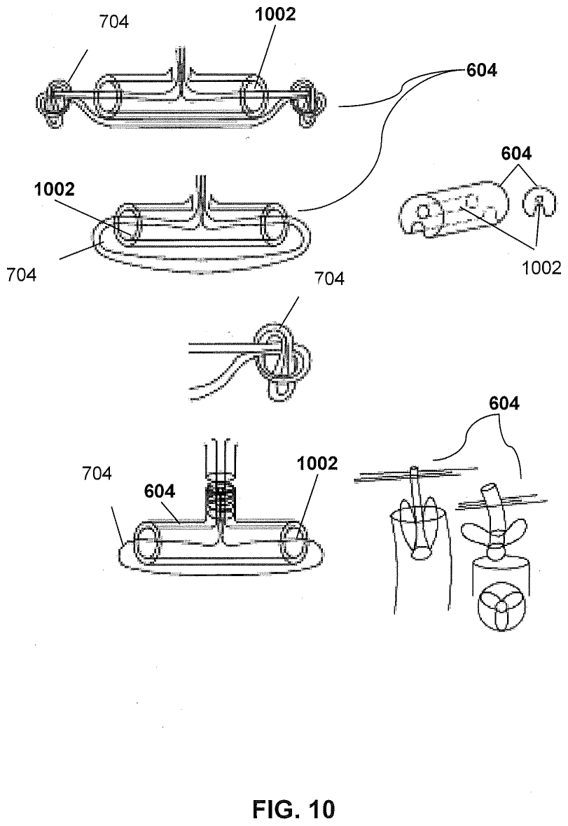

FIG. 10 shows anchor models, in accordance with an embodiment of the present disclosure. With reference to FIG. 10, the anchor devices 604 are one of: cylindrically shaped or rectangular mini-panels. In one embodiment, the system includes an anchor model that comprises a panel structure configured with tunnels 1002 for passage of ropes 704 Also, as shown in FIG. 10, the panel anchor 604 includes elongated, plate-like surfaces through which one or more ropes (cords) 704 can be inserted. The tunnels 1002 are integrated within the anchor 604 panel, allowing secure threading and fixation of the ropes 704 through the anchor 604 during or after deployment.

The tunnels 1002 are configured to prevent rope slippage or recoil, enabling a stable and tensioned connection between the anchor 604 and the rest of the support system 902 Such a design ensures that when traction is applied to the ropes 704, the anchor 604 remains securely positioned in the targeted tissue region, such as the ventricular groove or atrial wall, thereby maintaining the annular constriction effect intended by the support framework.

In another embodiment, the system includes an anchor 604 model comprising a screw 606 that incorporates a tunnel 1002 for passage of a rope 704 As further depicted in FIG. 10, the anchor 604 includes a helical or threaded screw 606 structure designed for insertion into cardiac tissue, such as the ventricular myocardium. The screw 606 may be advanced and fixed rotationally, allowing it to penetrate and remain embedded in the desired location of the heart.

The screw anchor 608 includes a built-in tunnel 1002 or channel, either passing through the shaft or adjacent to the screw body, through which the rope 704 is passed. Such a configuration allows the screw anchor 608 to act both as a mechanical fixation device and a tensioning anchor, ensuring that the rope 704 maintains its position under tension and contributes to the overall support and tightening effect on the mitral or tricuspid annulus.

In some embodiments, the system further includes a system of baskets 306 configured to trap a tip of a guidewire 404 in a left atrium, as described in FIG. 3. The system of baskets 306 is delivered through a venous transseptal guide catheter 212 introduced via the femoral vein, advanced into the right atrium, and then through a transseptal puncture 906 into the left atrium.

Once positioned in the left atrium, the system of baskets 306 is deployed to capture the guidewire tip that has been advanced from the left ventricle through the posterior sulcus and mitral annulus into the left atrial chamber. The guidewire 404 is exposed into the atrial cavity, and the baskets are configured to encircle and trap the wire tip, enabling the formation of a metallic arteriovenous loop 204 when the guidewire 404 is subsequently externalized via the venous catheter 212

The baskets may be symmetrical or asymmetrical, offering conformability to the anatomy of the left atrium and ensuring efficient wire capture regardless of positional variability, as shown in FIG. 3. The system of baskets 306 provides stabilization of the guidewire axis, allowing for controlled and reproducible deployment of the annuloplasty system components, including the anchors 604, the ropes 704, and the interconnecting bars 904

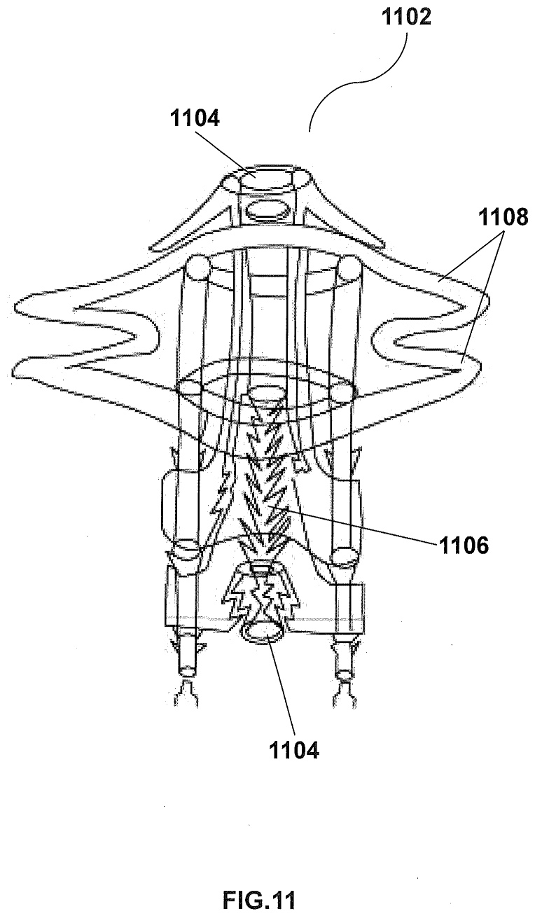

FIG. 11 shows the components of a central occluder 1102 without a central disk, in accordance with an embodiment of the present disclosure. In one embodiment, the system includes a central occluder 1102 positioned within the interatrial septum, wherein the central occluder 1102 is configured without a central disk. As illustrated in FIG. 11, the central occluder 1102 comprises two lateral occluding discs 1108, one positioned on the left atrial side and the other on the right atrial side of the septum, while the central portion of the device remains open or selectively collapsible to allow for integration of a support structure.

The central occluder's 1102 design allows for the passage of interventional devices, such as the ropes 704, the interconnecting bars 904, and other interlocking components such as interlocks 808 and interconnectors 812 through the septal region without interference from a rigid central disk. The central occluder 1102 is adapted to accommodate a passage mechanism or connector that aligns with the central support axis spanning the mitral annulus and interatrial septum.

In one embodiment, the central occluder 1102 comprises a lock for a zip 1106 More specifically, the central occluder 1102 includes an integrated locking mechanism 1106 which is adapted to engage with a zipper 806 element from the interconnecting bars 904 or support structure. This locking mechanism 1106 is designed to securely fasten the central portion of the annuloplasty framework once the bars 802 are zipped together across the mitral annulus.

The locking mechanism 1106 of the central occluder 1102 is situated in a central or near-central portion between the lateral discs 1108 and acts as a terminal anchor or fastener for the zipper 806 assembly, ensuring that the support structure 902 remains fixed in position, maintaining annular constriction and providing a reliable anchoring point within the septal wall.

In another embodiment, the central occluders 1102 comprise internal straight or curved zippers that trap the rope 704, either singular or in a group of ropes. The central occluder 1102 comprises a passage 1104, through which one or more elements of the annuloplasty system may pass. This passage 1104 may be a lumen or channel 1104 extending through the septal occluder, allowing the ropes 704 or bars 802 to traverse from the atrial to the ventricular side, or vice versa, without being obstructed. The passage 1104 is particularly advantageous in procedures where the central anchoring or interconnection of the support system 902 must align with the septal axis, and it ensures that no central disk interferes with the movement or alignment of the deployed structural components.

Conditional language such as, among others, “can,” “could,” “might” or “may,” unless specifically stated otherwise, are otherwise understood within the context as used in general to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Disjunctive language such as the phrase “at least one of X, Y, or Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

It will be understood by those within the art that, in general, terms used herein, are generally intended as “open” terms (e.g., the term “including” should be interpreted as “including but not limited to,” the term “having” should be interpreted as “having at least,” the term “includes” should be interpreted as “includes but is not limited to,” etc.).

What is claimed is:

1. A method for performing a mitral or tricuspid annuloplasty and creating a support system within a heart from which other medical procedures may be performed, the method comprising:
a. puncturing, using a guidewire from a catheter, a center of a posterior sulcus from a left ventricle to a left atrium, creating a central support point;
b. capturing the guidewire in the left atrium with an attachment device of a second catheter;
c. performing a puncture, using the guidewire, on each side of the central support point;
d. attaching anchor devices with ropes on each side of each of the puncture;
e. deploying the ropes attached to the anchor devices;
f. releasing interconnecting bars attached to the ropes;
g. coupling the interconnecting bars using a zipper; and
h. wherein the anchor devices, the ropes, and the interconnecting bars form the support system with a stable axis that incorporates both a mitral annulus and an interatrial septum.

2. The method of claim 1, further comprising placing a guide catheter in the left atrium by atrial transseptal puncture and in the left ventricle by arterial route from an aorta of the heart.

3. The method of claim 1, wherein the step of puncturing further comprises first puncturing the center of a posterior semicircle of the mitral annulus, which corresponds to a P2 segment of a mitral valve, and wherein a central catheter used for the puncture comprises two catheters.

4. The method of claim 1, wherein the attachment device of the second catheter is a telescoping system of a loop catheter, and the step of capturing further comprises trapping a guide tip in the left atrium with the telescoping system of the loop catheter.

5. The method of claim 1, wherein the second catheter capture attachment device comprises a system of baskets, and wherein the baskets are introduced via a venous catheter through auricular transeptal.

6. The method of claim 1, wherein performing a puncture, using the guidewire, on each side of the central support point comprises making lateral punctures at a constant distance from the support point using a central support catheter and a catheter parallel to the central support catheter.

7. The method of claim 1, wherein the anchor devices comprise screws and the step of attaching anchor devices with ropes on each side of each of the punctures comprises screwing to fix to the anchor devices.

8. The method of claim 1, wherein deploying the ropes attached to the anchor devices comprises pulling the ropes.

9. The method of claim 1, wherein the step of releasing the interconnecting bars attached to the ropes comprises unfolding the interconnecting bars as they exit the catheter, and wherein the unfolded interconnecting bars are attached to one another and are prevented from separating.

10. The method of claim 1, wherein the step of coupling the interconnecting bars using the zipper comprises constricting the mitral annulus with the bars.

11. A system for performing a mitral or tricuspid annuloplasty and creating a support system within a heart from which other medical procedures may be performed, the system comprises:
trans-atrial guide catheters;
an arterial retrograde catheter having a metallic guidewire including a tip, wherein the metallic guidewire is configured to puncture a center of a posterior sulcus from a left ventricle to a left atrium creating a central support point, wherein, after puncturing, the metallic guidewire tip is captured in the left atrium with an attachment device of a second catheter, and wherein the metallic guidewire is configured to perform a puncture on each side of the central support point;
anchor devices;
ropes attached to the anchor devices, wherein the anchor devices with ropes are attached on each side of the punctures; and
interconnecting bars attached to the ropes, wherein the interconnecting bars are coupled using a zipper,
wherein the anchor devices, the ropes, and the interconnecting bars form the support system, connected to the central support point, with a stable axis that incorporates both a mitral annulus and an interatrial septum.

12. The system of claim 11, further comprising a metallic loop, wherein the metallic loop is interconnected between the trans-atrial guide catheters and a trans-valvular mitral valve.

13. The system of claim 11, wherein the anchors include screws to prevent movement from a placed site.

14. The system of claim 11, wherein the interconnecting bars are attached to one another.

15. The system of claim 11, wherein the anchor devices are one of cylindrical shaped or rectangular mini-panels.

16. The system of claim 11, wherein the anchor devices have tunnels for the passage of the rope.

17. The system of claim 11, further comprising a system of baskets introduced through a catheter configured for capturing the metallic guidewire in the left atrium.

18. The system of claim 11, further comprising central occluders.

19. The system of claim 18, wherein the central occluders comprise internal straight or curved zippers that trap the rope, independently or in groups of ropes, with external cylindrical shapes.
